(19) United States
(12) Patent Application Publication
(10) Pub. No.: US 2026/0345443 A1
(43) Pub. Date: Nov. 5, 2026

(54) APPARATUS FOR CLEANING AN ENTERAL FEEDING TUBE

(71) Applicant: Becton, Dickinson and Company, Franklin Lakes, NJ (US)

(72) Inventors: Aniket Anand Kulkarni, Pune (IN); Shishir Prasad, Ramsey, NJ (US)

(73) Assignee: Becton, Dickinson and Company, Franklin Lakes, NJ (US)

(21) Appl. No.: 19/115,511

(22) PCT Filed: Oct. 31, 2022
(86) PCT No.: PCT/US2022/048409

§ 371 Date: Mar. 26, 2025

(51) Int. Cl. A61J 15/00; A61M 25/00; B08B 9/047

(52) U.S. Cl. CPC A61J 15/0026; B08B 9/047; A61M 2025/0019; B08B 2209/04

(57) ABSTRACT

Apparatus 30 for in-situ cleaning of a lumen of an enteral feeding tube in a patient. A flexible tubular conduit 32 is adapted for selective insertion and translation within the lumen. A mechanical, fluid-pressure powered actuator 50 is coupled to the distal end of the conduit, in fluid communication with an internal fluid passage 40 of the conduit. The actuator is configured to disrupt adhesion of debris occlusions within the lumen. A pressurized fluid source 42, such as a syringe, is coupled to the proximal end of the conduit, outside the patient's body, in fluid communication with the actuator, for selectively discharging pressurized fluid to and powering the actuator.

### CROSS-REFERENCE TO RELATED APPLICATIONS

This application is the National Stage entry of PCT/US22/48409, filed on Oct. 31, 2022, the disclosure of which is hereby incorporated by reference in its entirety.

### TECHNICAL FIELD

The present disclosure generally relates to apparatus for cleaning in situ, enteral feeding tubes. More particularly, the present disclosure relates to apparatus for de-clogging occlusions within such enteral feeding tubes, without having to withdraw the tube from the patient.

### BACKGROUND

Enteral nutrition involves delivery of nutrient formula or medicine to the gastrointestinal tract. Generally, where a patient requires short-term enteral nutrition, nutrients and food are administered to a patient's mouth by an enteral syringe, which may be referred to as an “oral syringe” or “oral delivery” of medication and does not require connection to a catheter or other device. When a patient cannot swallow food through the mouth or where long-term enteral nutrition is required, administration of nutrients and medicines to a patient can be accomplished with an enteral feeding system, assembly or device. Enteral feeding systems typically utilize catheters or tubes inserted into a patient's nose, mouth, stomach, duodenum or jejunum, through which nutrients are administered to the gastrointestinal tract. In percutaneous delivery methods, nutrients or medicines are delivered directly into the stomach, duodenum or jejunum of the gastrointestinal tract via a surgically implanted feeding tube/catheter. Specialized tubes/catheters are designed for delivery of nutrition and medicine to specific sites within the gastrointestinal system. For example, percutaneous endoscopic gastrostomy tubes (referred to as PEG tubes or G-tubes) are implanted into the stomach; jejunostomy tubes (J-tubes) are implanted into the small intestine; gastrojejunal (GJ-tubes) are implanted into the stomach and threaded into the small intestine; and nasogastric tubes (NG-tubes) are passed through the nose and esophagus into the stomach.

In all of the above-mentioned enteral delivery systems, an enteral syringe or another device is connected to the catheter to deliver the nutrients through the catheter. Enteral feeding catheters are distinguishable from intravenous catheters, which are inserted into the vasculature of patients to effect intravascular treatment, delivering medication through the circulatory or cardiovascular system by accessing any blood vessel. Such catheters include intravenous (IV) catheters, which are inserted into veins, and intra-arterial catheters, which are inserted into arteries.

Patients who have long-term feeding tube requirements are typically reside at home or at a nursing care facility. Over time, enteral feeding tubes/catheters are prone to clogging, despite various nutrient/medicine delivery, tube maintenance and de-clogging treatment protocols. Occlusions and other obstructions in such tubes must be cleared for continued successful enteral nutrient and medication delivery. Once a feeding tube clogs, it is typically prone to reclogging.

De-clogging protocols are time consuming, potentially delaying and impeding vital patient feeding and medicine administration for many hours. Known enteral tube de-clogging protocols include syringe irrigation and flushing of the tube with various liquids, including warmed or ambient temperature water, saline, acidic juices and carbonated solutions. Other known enteral tube de-clogging protocols involve injection of chemical solutions comprising acids, enzymes and other constituents to dissolve occlusions and decontaminate the tube. The specialized chemical solutions require up to hours of application time to treat and dissolve tube occlusions. Mechanical devices, such as brushes and jag-type scraper tips on flexible wires are passed through tube lumens to dislodge occlusions, but they are difficult to maneuver within tight bends and they are prone to breakage within the tube. The known brushes and scrapers do not remove all residual debris from the inner wall of the feeding tube lumen, which necessitates multiple, time-consuming, repetitive, sequential brushing and irrigation attempts and can also damage the lumen. Automated electromechanical tube de-clogging systems utilize motor-driven, spinning or reciprocating cleaning heads advanced through the tube lumen, with or without adjunct irrigation of the tube lumen. Such automated electromechanical systems are generally too complex and expensive for use at home residences or in nursing care facilities; they tend to be reserved for use in hospital facilities.

Despite best efforts, attempted de-clogging of an enteral feeding tube is not always successful. When an enteral tube/catheter cannot be cleaned to remove obstructions in a home or nursing care facility, it must be cleaned or replaced at hospital. A patient with an occluded enteral tube who resides at home or in nursing care facility must be transported to hospital for cleaning the tube by one of the more complex automated tube de-clogging systems or tube replacement. Percutaneous implanted, enteral tubes/catheters require surgery for replacement.

It is preferable for patient wellness and comfort, as well as for delivery of effective, lower cost medical services to develop an enteral tube/catheter cleaning system which effectively de-clogs occlusions inexpensively and is usable by care providers at in-home and in skilled nursing patient care environments.

### SUMMARY

In one or more embodiments, an apparatus disclosed herein cleans the lumen of an enteral feeding tube or catheter in situ within a patient, removing occlusions and other obstructions. This apparatus is suitable for use in gastrostomy-, jejunostomy- and nasogastric-type enteral feeding tubes/catheters. A flexible tubular conduit of the cleaning apparatus is adapted for selective insertion and translation within the lumen. A mechanical, fluid-pressure powered actuator is coupled to the distal end of the conduit, in fluid communication with an internal fluid passage of the conduit. The mechanical actuator disrupts adhesion of debris occlusions within the lumen. A pressurized fluid source, such as a syringe, is coupled to the proximal end of the conduit, outside the patient's body, in fluid communication with the actuator, for selectively discharging pressurized fluid to and providing stored energy to power the actuator. Where the pressurized fluid source is a common syringe, the entire cleaning apparatus is simple to operate, and suitable for home use or nursing care facilities by clinicians and patient care givers. The fluid-pressure powered, mechanical actuator cleans feeding tube lumens quickly and efficiently, at low cost. Successful tube de-clogging with the disclosed apparatus eliminates need to withdraw and replace the tube/catheter from the patient, which might otherwise be required if tube cleaning was unsuccessful or unsatisfactory. Elimination of catheter/tube replacement avoids patient discomfort and costs associated replacement. For example, successful de-clogging of a feeding tube in a home or skilled nursing care facility avoids need to transport the patient to a hospital for more specialized cleaning attempts or tube replacement.

One aspect of the present disclosure pertains to apparatus for in-situ cleaning of a lumen of an enteral feeding catheter/tube in a patient with an inner wall surface of the lumen defining an inner diameter. The apparatus comprises a flexible, tubular conduit adapted for selective insertion and translation within the lumen. The conduit has distal and proximal ends, an outer diameter smaller than the inner diameter of the lumen, and an internal fluid passage for establishing fluid communication between its distal and proximal ends. A mechanical, fluid-pressure powered actuator is coupled to the distal end of the conduit, in fluid communication with the conduit's internal fluid passage. The actuator disrupts adhesion of debris occlusions on the inner wall surface of the of the lumen. A pressurized fluid source is coupled to the proximal end of the conduit, in fluid communication with the actuator, for selectively discharging pressurized fluid to and powering the actuator. In some embodiments, the pressurized fluid source is a plunger actuated syringe, while in other embodiments, the pressurized fluid source is a pump or an externally pressurized bladder. In some embodiments, the driven actuator rotates to break occlusions in the lumen, while in other embodiments the actuator inflates to break occlusions in the lumen.

Another aspect of the present disclosure pertains to apparatus for in-situ cleaning of a lumen of an enteral feeding catheter/tube in a patient, with an inner wall surface of the lumen defining an inner diameter. The apparatus includes a flexible tubular conduit, having respective distal and proximal ends, and an internal fluid passage for establishing fluid communication between its distal and proximal ends. A mechanical, fluid-pressure powered actuator is coupled to the distal end of the conduit, in fluid communication with the latter's internal fluid passage. This actuator has a housing, with a distal end coupled to a proximal axial end of a rotating rotor. The rotating rotor has a turbine on its proximal end, in communication with the fluid passage of the conduit. Blades are disposed on a distal end of the rotating rotor, external the housing, for disrupting debris occlusions. The turbine of the rotating rotor is powered by pressurized fluid discharged from a pressurized fluid source that is coupled to the proximal end of the conduit, in fluid communication with the actuator, for selectively rotating the rotating rotor and its blades.

Other aspects of the present disclosure pertain to apparatus for in-situ cleaning of a lumen of an enteral feeding tube in a patient, with an inner wall surface of the lumen defining an inner diameter. The cleaning apparatus includes a flexible tubular conduit, having respective distal and proximal ends, and an internal fluid passage for establishing fluid communication between its ends. A mechanical, fluid-pressure powered actuator is coupled to the distal end of the conduit. This actuator has inflatable bellows, with an internal cavity in communication with the internal fluid passage. The bellows has an inflated outer diameter and/or inflated axial length greater than its respective uninflated outer diameter and/or axial length. A pressurized fluid source is coupled to the proximal end of the conduit, in fluid communication with the internal fluid passage and the internal cavity of the bellows. The pressurized fluid, when discharged into the bellows selectively inflates the bellows.

### BRIEF DESCRIPTION OF THE DRAWINGS

Exemplary embodiments of the disclosure are further described in the following detailed description in conjunction with the accompanying drawings, in which:

FIG. 1 is a schematic view of an exemplary enteral feeding tube cleaning apparatus of the present disclosure inserted within a percutaneous endoscopic gastrostomy (PEG) tube in situ the stomach of a patient, while de-clogging an occlusion within the PEG tube;

FIG. 1A is an enlarged view of the area 1A in FIG. 1;

FIG. 2 is a perspective view of the of enteral feeding tube cleaning apparatus FIG. 1;

FIG. 3 is a perspective view of the enteral feeding tube cleaning apparatus of FIGS. 1 and 2;

FIG. 4 is a cross-sectional view of the actuator of the enteral feeding tube cleaning apparatus of FIGS. 1-3;

FIG. 5 is a perspective, distal end view of the rotating rotor of FIG. 4;

FIG. 6 is a perspective, proximal end view of the rotating rotor of FIG. 4;

FIG. 7 is a plan view of the proximal end of the rotating rotor of FIG. 4;

FIG. 8 is a distal end, perspective view of another embodiment of an exemplary enteral feeding tube cleaning apparatus of the present disclosure;

FIG. 9 is a partial elevational cross-sectional view of the actuator of the enteral feeding tube cleaning apparatus of FIG. 8 in a deflated condition;

FIG. 10 is a partial elevational cross-sectional view of the actuator of the enteral feeding tube cleaning apparatus of FIG. 8 in an inflated condition;

FIG. 11 is another partial elevational cross-sectional view of the actuator of the enteral feeding tube cleaning apparatus of FIG. 8 in an inflated condition;

FIG. 12 is a schematic view of the enteral feeding tube cleaning apparatus of FIGS. 8-11 of the present disclosure inserted within a percutaneous endoscopic gastrostomy (PEG) tube in situ the stomach of a patient, while de-clogging an occlusion within the PEG tube;

FIG. 13 is a schematic view of an additional embodiment of an enteral feeding tube cleaning apparatus of the present disclosure;

FIG. 14 is a schematic view of another additional embodiment of an enteral feeding tube cleaning apparatus of the present disclosure; and

FIG. 15 is a process flow chart illustrative of a method for in situ cleaning, de-clogging and clearing of an occlusion or other obstruction within an in situ enteral feeding tube.

To facilitate understanding, identical reference numerals have been used, where possible, to designate identical elements that are common to the figures. The figures are not drawn to scale.

### DETAILED DESCRIPTION

Aspects of the apparatus disclosed herein cleans the lumen of an enteral feeding tube or catheter in situ within a patient, removing occlusions and other obstructions. A flexible tubular conduit of the cleaning apparatus is adapted for selective insertion and translation within the lumen. A mechanical, fluid-pressure powered actuator is coupled to the distal end of the conduit, in fluid communication with an internal fluid passage of the conduit. In some embodiments, the mechanical actuator utilizes rotating blades to dislodge and remove debris and other occlusions from lumens of feeding catheter/stubes. In other embodiments, the mechanical actuator utilizes an inflatable bladder such as bellows to dislodge and remove debris and other occlusions from tube lumens. The mechanical actuator is configured to disrupt adhesion of debris occlusions within the lumen. A pressurized fluid source, such as a syringe, is coupled to the proximal end of the conduit, outside the patient's body, in fluid communication with the actuator, for selectively discharging pressurized fluid to and powering the actuator. The fluid-pressure powered, mechanical actuator cleans feeding tube lumens quickly and efficiently.

Before describing several exemplary embodiments of the disclosure, it is to be understood that the disclosure is not limited to the details of construction or process steps set forth in the following description. The disclosure is capable of other embodiments and of being practiced or being conducted in many ways. The matters exemplified in this description are provided to assist in a comprehensive understanding of exemplary embodiments of the disclosure. Also, descriptions of well-known functions and constructions are omitted for clarity and conciseness.

With respect to terms used in this disclosure, the following definitions are provided. As used herein, the use of “a,” “an,” and “the” includes the singular and plural. In this disclosure, a convention is followed wherein the distal end of the device is the end closest to a patient and the proximal end of the device is the end away from the patient and closest to a clinician.

As used herein, the term “Luer connector” refers to a connection collar that is the standard way of attaching syringes, catheters, hubbed needles, IV tubes, etc. to each other. The Luer connector consists of male and female interlocking tubes, slightly tapered to hold together better with even just a simple pressure/twist fit. Luer connectors can optionally include an additional outer rim of threading, allowing them to be more secure. The Luer connector male end is generally associated with a syringe and can interlock and connect to the female end located a needle hub.

As used herein, ISO 80369-7:2016 defines a specification for standard Luer connectors including a 6% taper between the distal end and the proximal end. A male standard luer connector increases from the open distal end to the proximal end. A female standard luer connector decreases from the open proximal end to the distal end. According to ISO 80369-7:2016, a male standard Luer connector has an outer cross-sectional diameter measured 0.75 mm from the distal end of the tip of between 3.970 mm and 4.072 mm. The length of the male standard luer taper is between 7.500 mm to 10.500 mm. The outer cross-sectional diameter measured 7.500 mm from the distal end of the tip is between 4.376 mm and 4.476 mm. As used herein, the phrases “male standard Luer connector” and “female standard Luer connector” shall refer to connectors having the dimensions described in ISO 80369-7, which is hereby incorporated by reference in its entirety.

As would be readily appreciated by skilled artisans in the relevant art, while descriptive terms such as “tip”, “hub”, “thread”, “protrusion/insert”, “tab”, “slope”, “wall”, “top”, “side”, “bottom” and others are used throughout this specification to facilitate understanding, it is not intended to limit any components that can be used in combinations or individually to implement various aspects of the embodiments of the present disclosure.

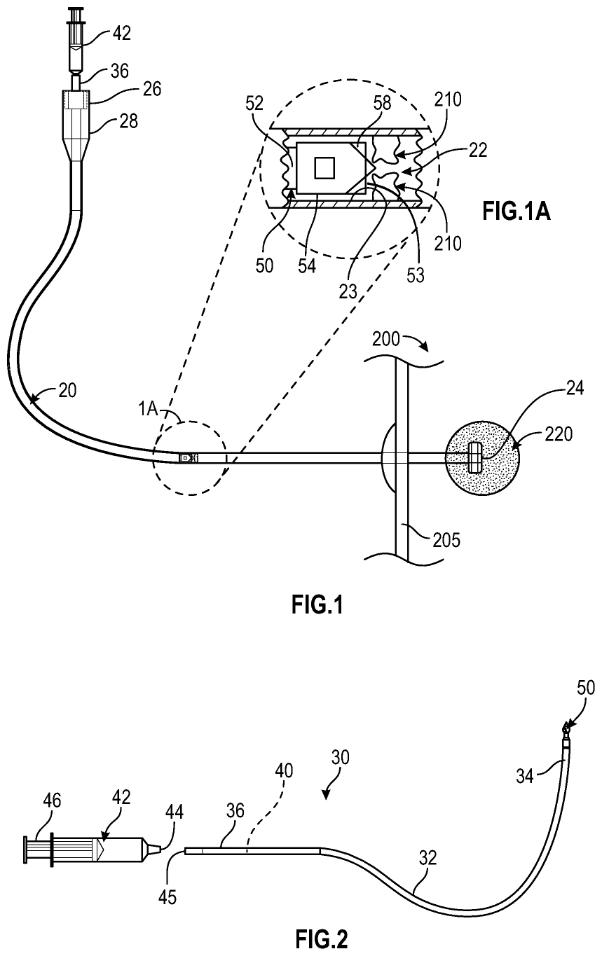

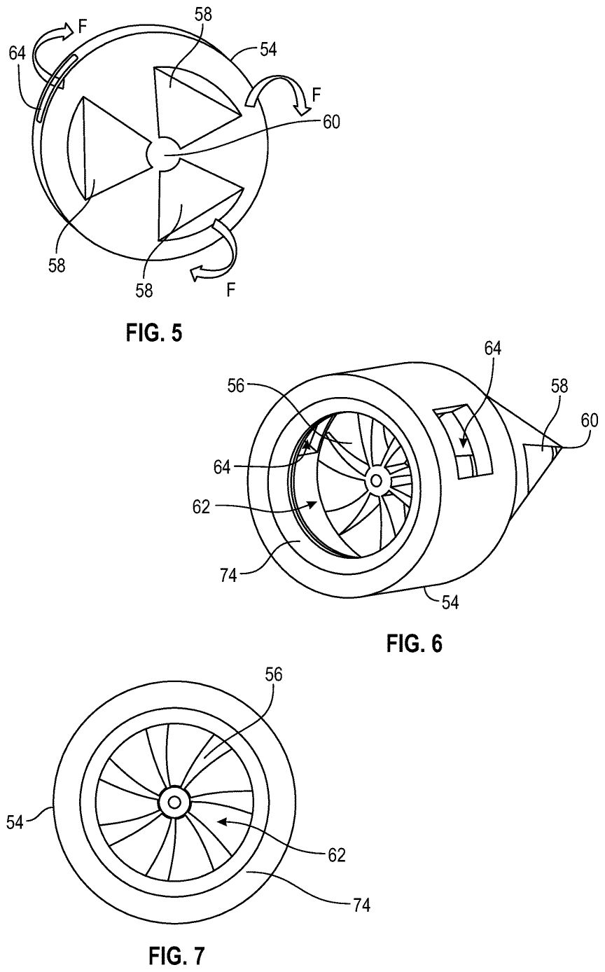

The following non-limiting examples demonstrate principles according to one or more embodiments of the disclosure. Referring now to the drawings, a first aspect of the present disclosure is shown in FIGS. 1-7. In FIG. 1, a percutaneous endoscopic gastrostomy (PEG)-type, enteral feeding tube 20 has a lumen 22 defined by an inner wall 23 of the tube. A distal end 24 of the enteral feeding tube 20 is shown inserted within a stomach 220 of a patient 200. A proximal end 26 of the enteral feeding tube 20 includes an ENFit compatible feeding port 28 that is compliant to ISO 80369-3 standard and is not connectible to a male or female standard Luer connector. Connection incompatibility between ENFit fittings and standard Luer connector fittings prevents misadministration of an enteral feeding or medication by the wrong route. In FIG. 1, the enteral feeding tube 20 has an occlusion or obstruction O of nutrient or drug debris adhering to its inner wall 23. A cleaning apparatus 30 of the present disclosure is inserted and advanced within the lumen 22 of the enteral feeding tube 20, translated in proximity to the occlusion O.

Referring to FIGS. 1 and 2, the cleaning apparatus 30 comprises a conduit 32 which is flexible with a distal end 34 and a proximal end 36. An inner wall 38 of the conduit 32 defines an internal fluid passage 40 throughout its entire length, for establishing fluid communication between its distal end 34 and proximal end 36. The conduit 32 is constructed with medical-grade tubing. A syringe 42 has a male luer connector 44 coupled to a female luer connector 45 within the proximal end 36 of the conduit 32. Use of the male luer connector 44 and the female luer connector 45 prevents misconnection of an ENFit connector to the conduit 32. Depression of plunger 46 of the syringe 42 discharges pressurized fluid, such as sterile water or saline solution or other desired fluid, from the male luer connector 44. An actuator 50, which is a fluid pressure powered actuator in some embodiments, provides a mechanical actuator, which is coupled to the distal end 34 of the conduit 32, in fluid communication with the internal fluid passage 40 and the syringe 42. Pressurized fluid discharged by the syringe 42 powers the actuator 50 and de-clogs the occlusion O.

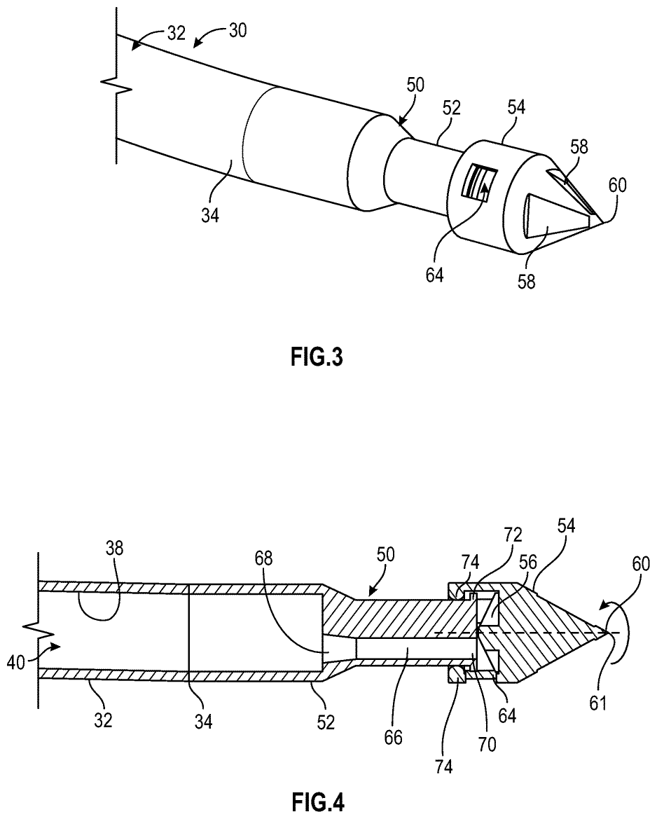

FIGS. 3-7 show structure of the actuator 50. The actuator 50 comprises a housing 52 and a rotating rotor 54. The rotating rotor 54 has a turbine 56 on its proximal end, is in fluid communication with the internal fluid passage 40. The turbine 56 is powered by pressurized fluid discharged from the syringe 42, which spins and rotates the rotating rotor 54. In other embodiments, other pressurized fluid sources power the turbine 56 and rotate the rotating rotor 54. Rotating blades 58 on a distal end 60 of the rotating rotor 54, external the housing 52, disrupt occlusions 210 in the form of debris on the inner wall 23 of the lumen 22, thereby cleaning the enteral feeding tube 20 while still implanted, in situ, in the patient 200 (FIG. 1) through the skin 205 of the patient 200. The distal end 60 of the rotating rotor 54 has a conical profile distal tip 61 to facilitate translation and advancement into the lumen 22 of the enteral feeding tube 20 (FIG. 1) with the blades 58 projecting axially and radially therefrom relative to a rotational axis of the rotating rotor.

FIGS. 4-5 show the pressurized fluid F pathway between the internal fluid passage 40 and the actuator 50. The rotating rotor 54 has an intake port 62 and a plurality of circumferentially oriented outlet ports 64. The housing 52 defines a nozzle 66, with a nozzle inlet 68 in communication with the internal fluid passage 40 of the conduit 32 and a nozzle outlet 70 for directing pressurized fluid onto the turbine 56. The proximal end of the rotating rotor 54 is coupled rotatively to a distal axial end 53 of the housing 52 by corresponding mating, male and female annular flanges. In the embodiment of FIGS. 4-5, the distal axial end 53 of the housing defines a male annular flange 72 and the proximal axial end of the rotating rotor defines a mating female annular flange 74. In other embodiments, the male and female flanges are reversed, so that the rotating rotor defines the male flange, and the housing defines the female flange. Pressurized fluid F discharged out of the outlet ports 64 of the rotating rotor 54 irrigates the lumen 22 of the enteral feeding tube 20, which assists liquefaction and dissolving of occlusions 210.

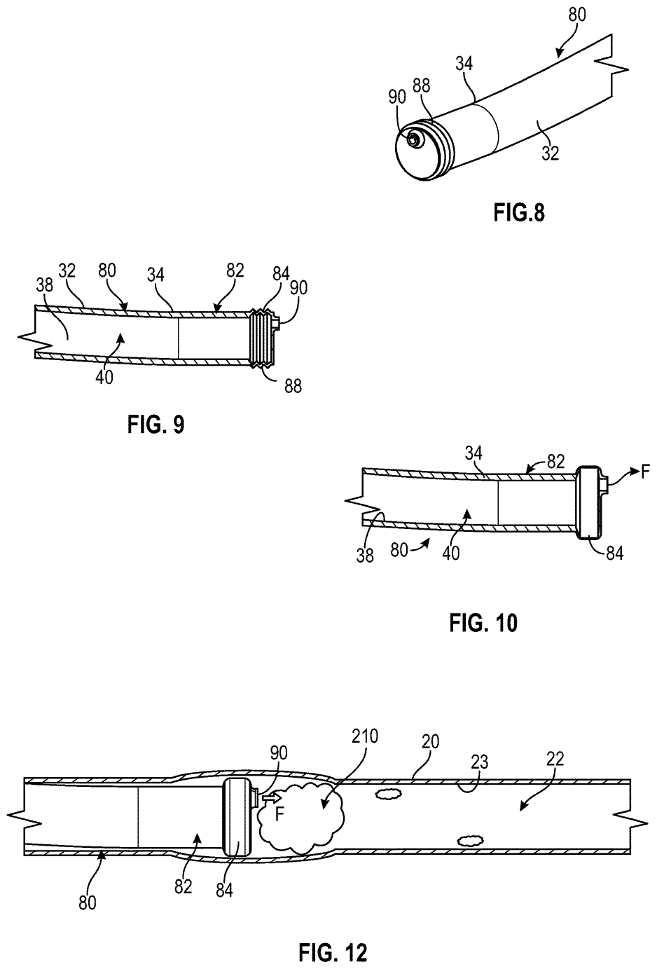

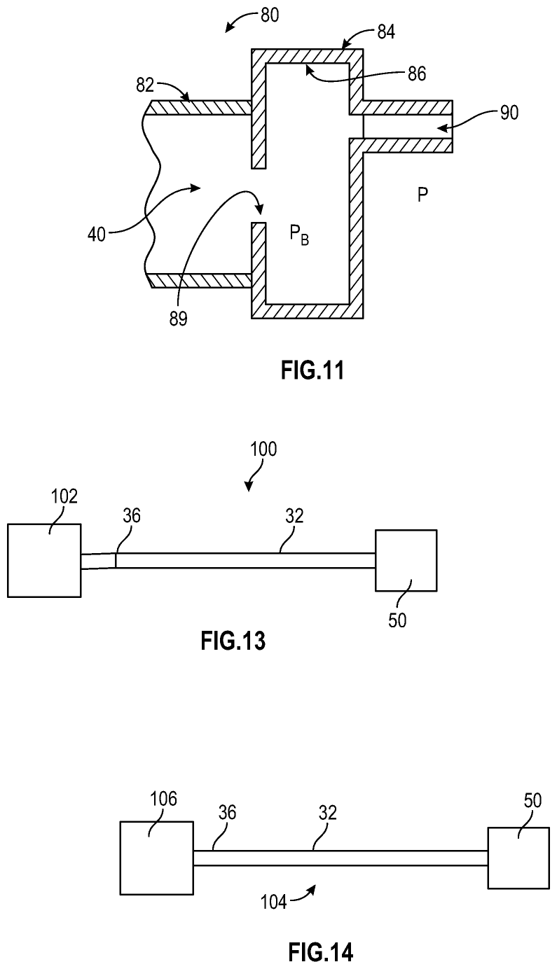

Another aspect of the present invention is depicted in FIGS. 8-12, wherein the cleaning apparatus 80 has a fluid-pressure powered, inflatable-type mechanical actuator; more specifically, an inflatable bellows-type inflatable actuator 82. The bellows 84 define an internal cavity 86 that is in fluid communication with the internal fluid passage 40 of the conduit 32 by the bellows inlet 89. The bellows 84 is inflatable by discharging pressurized fluid F from the pressurized fluid source, such as the syringe 42 of FIG. 1, so that pressure PB in the bellows is greater than ambient pressure P in the internal fluid passage 40 of the enteral feeding tube 20. The bellows 84 of the embodiment of FIGS. 8-12, have inflatable pleats 88, providing a pleated construction. Comparing FIGS. 9 and 10, the inflated outer diameter and/or inflated axial length of the bellows 84 (FIG. 10) is greater than its respective uninflated outer diameter and/or axial length (FIG. 9). As shown in FIG. 12, inflation of the bellows 84 increases internal diameter of the lumen 22 defined by the distance between opposing ends of the inner wall 23 of the enteral feeding tube 20, thereby stretching the latter's flexible wall and dislodging occlusions 210 within the lumen. Pressurized fluid F discharged out of the outlet port 90 of the bellows 84 irrigates the lumen 22 of the enteral feeding tube 20, which assists liquefaction, dissolving and transport of occlusions 210 downstream into the patient's stomach.

While the embodiments the cleaning apparatus 30 and cleaning apparatus 80 of FIGS. 1-12 so far have identified use of a syringe 42 as the pressurized fluid F source, other embodiments employ other types of pressurized fluid sources. In the embodiment of FIG. 13, the cleaning apparatus 100 utilizes a pump 102, in fluid communication with the actuator 50, via the conduit 32, for its pressurized fluid source. In the embodiment of FIG. 14, the cleaning apparatus 104 utilizes an externally pressurized bladder 106, in fluid communication with the actuator 50, via the conduit 32, for its pressurized fluid source.

The components of the exemplary cleaning apparatuses 30, 80, 100 and 104, are fabricated with a variety of materials suitable for medical and health care applications. For example, embodiments of the conduit 32, syringe 42 and actuator 50 and bellows 84 are fabricated from a medical-grade material, including but not limited to nylon, polypropylene, polycarbonate, polyvinylidene fluoride, acrylonitrile butadiene styrene, polyvinyl chloride, thermoplastic elastomers and thermoset rubber.

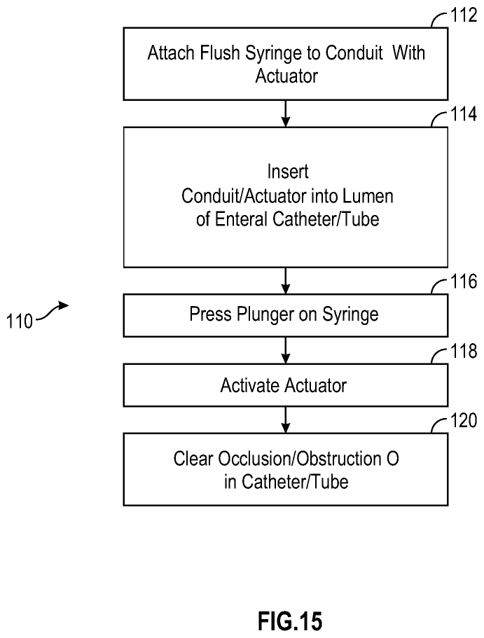

FIG. 15 depicts the process steps 110 for utilizing the various embodiments of cleaning apparatuses 30, 80, 100, 104 of this disclosure for cleaning a lumen of an enteral feeding catheter/tube, including removal and dislodging of debris, occlusions and other obstructions within the lumen. Exemplary types of enteral feeding tubes suitable for cleaning with cleaning apparatus of this disclosure include, without limitation, percutaneous endoscopic gastrostomy tubes (referred to as PEG tubes or G-tubes); jejunostomy tubes (J-tubes); gastrojejunal (GJ-tubes); and nasogastric tubes (NG-tubes). The cleaning apparatus embodiments of this disclosure are suitable for cleaning occlusions and other obstructions from lumens of other types of tubes and catheters.

For brevity, the process steps 110 are describe using the cleaning apparatus 30 of FIGS. 1-7, though the same process is useable for any of the other apparatus embodiments. The pressurized fluid source, such a syringe 42 filled with water or saline, is attached to the conduit 32 by mating the male luer connector 44 to the female luer connector 45 at step 112. The cleaning apparatus 30, comprising the coupled actuator 50, conduit 32 and syringe 42 are inserted, translated, and advanced into the lumen 22 of the enteral feeding tube 20, proximate the occlusion/obstruction O, by a clinician, step 114. The clinician then presses the plunger 46 of the syringe 42 (or activates any alternative pressure sources that are coupled to the conduit 32), step 116. Pressing the syringe plunger 46, activates the actuator 50, by causing the pressurized fluid F to spin the turbine 56, rotating its rotor 54, step 118. The blades 58 of the rotating rotor 54 disrupt occlusions 210 in the form of debris within the lumen 22 of the enteral feeding tube 20, step 120, thereby cleaning the enteral tube. The pressurized fluid F, disbursed from the outlet ports 64, also irrigates the lumen 22 and acts as fluid lubricant carrying away the clog debris from the now cleaned obstruction site.

Reference throughout this specification to “one embodiment,” “certain embodiments,” “various embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in various embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein provided a description with reference to particular embodiments, it is to be understood that these embodiments are merely illustrative of the principles and applications of the disclosure. It will be apparent to those skilled in the art that various modifications and variations can be made to the present disclosure without departing from the spirit and scope thereof. Thus, it is intended that the present disclosure include modifications and variations that are within the scope of the appended claims and their equivalents.

What is claimed is:

1. An apparatus for in-situ cleaning of a lumen of an enteral feeding tube in a patient, an inner wall surface of the lumen defining an inner diameter, comprising:
a flexible tubular conduit adapted for selective insertion and translation within the lumen, the conduit having a distal end and a proximal end, an outer diameter smaller than the inner diameter of the lumen, and an internal fluid passage for establishing fluid communication between the distal end and the proximal end;
a mechanical, fluid-pressure powered actuator coupled to the distal end of the conduit, in fluid communication with the internal fluid passage thereof, the actuator configured to disrupt adhesion of debris occlusions on the inner wall surface of the of the lumen; and
a pressurized fluid source coupled to the proximal end of the conduit, in fluid communication with the actuator, for selectively discharging pressurized fluid to and powering the actuator.

2. The apparatus of claim 1, the pressurized fluid source comprising a plunger actuated syringe.

3. The apparatus of claim 1, the pressurized fluid source comprising a pump.

4. The apparatus of claim 1, the pressurized fluid source comprising an externally pressurized bladder.

5. The apparatus of claim 1, the actuator further comprising:
a housing having an outer diameter smaller than the inner diameter of the lumen, a rotating rotor coupled to the housing, the rotating rotor having a turbine on a proximal end thereof in communication with the fluid passage, the turbine powered by pressurized fluid discharged from the pressurized fluid source, and the rotating rotor having rotating blades on a distal end thereof external the housing, for disrupting debris occlusions on the inner wall surface of the lumen.

6. The apparatus of claim 5, the pressurized fluid source comprising a plunger actuated syringe.

7. The apparatus of claim 1, the actuator further comprising bellows with an internal cavity in communication with the fluid passage, the bellows, configured to inflated by pressurized fluid discharged from the pressurized fluid source, the bellows having an inflated outer diameter and/or inflated axial length greater than its respective uninflated outer diameter and/or axial length.

8. The apparatus of claim 7, the bellows having an inflated outer diameter greater than the inner diameter of the lumen.

9. An apparatus for in-situ cleaning of a lumen of an enteral feeding tube in a patient, an inner wall surface of the lumen defining an inner diameter, comprising:
a flexible tubular conduit, having a distal end and a proximal end, and an internal fluid passage for establishing fluid communication between the distal and proximal ends;
a mechanical, fluid-pressure powered actuator coupled to the distal end of the conduit and in fluid communication with the internal fluid passage, the mechanical, fluid-pressure powered actuator having a housing having a rotating rotor having a proximal axial end coupled to a distal axial end of the housing, the rotating rotor having a turbine on its proximal end in communication with the fluid passage, and the rotating rotor having blades on a distal end thereof external the housing, for disrupting debris occlusions; and
a pressurized fluid source coupled to the proximal end of the conduit, in fluid communication with the actuator, for selectively rotating the rotating rotor by discharging pressurized fluid to and powering the turbine.

10. The apparatus of claim 9, further comprising the rotating rotor having an intake port in fluid communication with the internal fluid passage and the turbine; and an outlet port for discharging the pressurized fluid.

11. The apparatus of claim 10, the rotating rotor having a conical profile distal tip with the blades projecting axially and radially therefrom relative to a rotational axis of the rotating rotor.

12. The apparatus of claim 11, the housing further comprising a nozzle having an inlet on its proximal end in fluid communication with the internal fluid passage and an outlet on a distal end of the housing in communication with the intake port of the rotating rotor.

13. The apparatus of claim 12, further comprising the distal axial end of the housing and the proximal end of the rotating rotor respectively having corresponding mating, male and female annular flanges rotatively coupled to each other, and the outlet port circumferentially oriented about the rotating rotor.

14. The apparatus of claim 9, the pressurized fluid source comprising a plunger actuated syringe.

15. Apparatus for in-situ cleaning of a lumen of an enteral feeding tube in a patient, an inner wall surface of the lumen defining an inner diameter, comprising:
a flexible tubular conduit, having a distal end and a proximal end, and an internal fluid passage for establishing fluid communication between the distal and proximal ends;
a mechanical, fluid-pressure powered actuator coupled to the distal end of the conduit, having inflatable bellows with an internal cavity in communication with the internal fluid passage, the bellows having an inflated outer diameter and/or inflated axial length greater than its respective uninflated outer diameter and/or axial length; and
a pressurized fluid source coupled to the proximal end of the conduit, in fluid communication with the internal fluid passage and the internal cavity of the bellows, for selectively inflating the bellows by discharging pressurized fluid therein.

16. The apparatus of claim 15, the bellows having pleated construction.

17. The apparatus of claim 16, the bellows having an outlet port on a distal axial end thereof, for discharging pressurized fluid out of the actuator.

18. The apparatus of claim 17, the pressurized fluid source comprising a plunger actuated syringe.

19. The apparatus of claim 15, the bellows having an outlet port on a distal axial end thereof, for discharging pressurized fluid out of the actuator.

20. The apparatus of claim 19, the pressurized fluid source comprising a plunger actuated syringe.
